(19) United States
(12) Patent Application Publication
(10) Pub. No.: US 2026/0096673 A1
(43) Pub. Date: Apr. 9, 2026

(54) VISUALIZATION UNIT

(71) Applicant: Morgan's Holding Global Ltd, London (GB)

(72) Inventor: Rajeev Lee, East Sussex (GB)

(21) Appl. No.: 19/053,970

(22) Filed: Feb. 14, 2025

Related U.S. Application Data
(60) Provisional application No. 63/703,657, filed on Oct. 4, 2024.

(51) Int. Cl. A47F 5/00; A61L 9/015; B08B 13/00; G06Q 30/0601; G06V 20/52; G06V 40/10

(52) U.S. Cl. CPC A47F 5/0043; A61L 9/015; B08B 13/00; G06V 20/52; G06V 40/10; A61L 2209/111; A61L 2209/13; A61L 2209/15; G06Q 30/0633

(57) ABSTRACT

A visualization unit that can include a standing wall, ornamental shelving on the standing wall, one or more actuating devices, and one or more digital labels. And methods, systems, and apparatus, including computer programs encoded on computer storage media, for a visualization unit. One of the methods includes processing sensor data to determine whether one or more conditions are satisfied; in response to determining that one or more conditions are satisfied, generating a signal configured to cause one or more actuating devices to actuate; and transmitting the generated signal to the one or more actuating devices.

### CROSS-REFERENCE TO RELATED APPLICATIONS

This application claims the benefit of U.S. Provisional Application No. 63/703,657, filed Oct. 4, 2024, the contents of which are incorporated by reference herein.

### BACKGROUND

Visualization units can include systems or devices that render graphical representations of data. Users of visualization units can interact with units using one or more interfaces, such as touch screen input on the visualization units or connected devices.

### SUMMARY

In general, one innovative aspect of the subject matter described in this specification can be embodied in a visualization unit that includes a standing wall disposed between a first screen and a second screen, wherein the first screen covers a first face of the standing wall and the second screen covers a second face of the standing wall, wherein the first face is opposite the second face; the standing wall having ornamental shelving partially obscuring the first screen and the second screen and defining rows along the first screen and the second screen; an actuating device mechanically coupled to the standing wall; and a digital label along an outer edge of the ornamental shelving configured to display an identifier of an object represented on an unobscured portion of either the first screen or the second screen.

The foregoing and other embodiments can each optionally include one or more of the following features, alone or in combination. In particular, one embodiment includes all the following features in combination. Feature 1: The actuating device is mechanically coupled to the standing wall along an outer frame of at least one of the first face of the second face of the standing wall. Feature 2: The actuating device includes at least one of a scent dispenser, a motion detector, a communication element, or a camera. Feature 3: The ornamental shelving does not hold objects displayed on the first screen. Feature 4: The standing wall houses one or more elements providing electricity or resources to the actuating device. Feature 5: The standing wall houses one or more scent reservoirs used by a scent dispenser. Feature 6: The actuating device is controlled by one or more computers configured to receive image data, process the image data to determine whether a person is approaching the visualization unit, and, in response to determining that a person is approaching the visualization unit, transmit a signal to the actuating device to cause the actuating device to disperse a scent. Feature 7: The actuating device is embedded within the standing wall. Feature 8: The standing wall includes a permeable cover to house the actuating device within the standing wall.

In general, one innovative aspect of the subject matter described in this specification can be embodied in methods that include the actions of receiving sensor data from one or more sensors of a visualization unit; storing the sensor data within memory of the visualization unit; processing the sensor data to determine whether one or more conditions are satisfied; in response to determining that one or more conditions are satisfied, generating a signal configured to cause one or more actuating devices to actuate; and transmitting the generated signal to the one or more actuating devices. Other embodiments of this aspect include corresponding computer systems, apparatus, and computer programs recorded on one or more computer storage devices, each configured to perform the actions of the methods.

The foregoing and other embodiments can each optionally include one or more of the following features, alone or in combination. In particular, one embodiment includes all the following features in combination. Feature 1: Receiving sensor data from the one or more sensors of the visualization unit comprises: receiving image data from a camera mechanically coupled to the visualization unit. Feature 2: Receiving sensor data from the one or more sensors of the visualization unit comprises: receiving proximity data from a proximity sensor mechanically coupled to the visualization unit. Feature 3: Receiving sensor data from the one or more sensors of the visualization unit comprises: receiving a signal transmitted by a mobile device. Feature 4: Receiving sensor data from the one or more sensors of the visualization unit comprises: scanning a scanning element provided by a mobile device. Feature 5: Receiving sensor data from the one or more sensors of the visualization unit comprises: receiving data indicating an interaction of a user selecting an object displayed on the visualization unit. Feature 6: Storing the sensor data within memory of the visualization unit comprises: storing the received data within data representing a virtual basket associated with the user. Feature 7: Generating the signal configured to cause the one or more actuating devices to actuate comprises: generating a signal configured to cause a scent dispenser to dispense a scent compound. Feature 8: Generating the signal configured to cause the scent dispenser to dispense the scent compound comprises: generating a signal configured to instruct the scent dispenser to obtain a scent compound from a particular scent reservoir from a set of one or more scent reservoirs housed in the visualization unit. Feature 9: Generating the signal configured to cause the one or more actuating devices to actuate comprises: generating a signal configured to cause a cleaning element to clean an area of the visualization unit. Feature 10: Generating the signal configured to cause the cleaning element to clean the area of the visualization unit occurs in response to detecting a person is not within a distance of the visualization unit. Feature 11: Generating the signal configured to cause the one or more actuating devices to actuate comprises: generating a signal configured to change an operating mode of the visualization unit. Feature 12: Generating the signal configured to change the operating mode of the visualization unit comprises generating a turn off signal or sleep signal. Feature 13: Generating the signal configured to change the operating mode of the visualization unit occurs in response to determining a current time of day satisfies a threshold time of day for changing the operating mode.

The technology described in this specification can be implemented so as to realize one or more of the following advantages. For example, visualization units can be used to display objects for sale in a store or outside. A visualization unit offers improvements for stores by eliminating or reducing physical items needed to be stockpiled, thereby reducing a required footprint of the store and spoilage. Store footprint can be reduced by the slender form factor of visualization units (compared to existing object shelving) which do not have to store rows and rows of tangible objects, and by decreasing duplicate shelving space traditionally used to increase stock of particular items.

In addition, especially in high crime areas, visualization units can solve the urgent need of allowing stores to operate without allowing rampant theft to cause store closures. Rampant theft of goods has caused some communities to lose vital shopping areas. A visualization unit can solve the problem of theft by allowing customers to shop and purchase an item without storing the item at the physical store. Instead, objects can be delivered to the person at their home, place of business, or other specified location. Visualization units can be used as a sole means of object visualization or in addition to one or more tangible item visualizations, such as a traditional shopping store aisle. As such, visualization units can be used to prevent theft of higher value items while traditional units with tangible items can be used to store lower value items. Removal of physical items can also help reduce the spread of germs or viruses which can spread through contact with physical items in traditional stores. Periodic, automized, sanitization can also help reduce the spread of germs or viruses.

A visualization unit can also improve a user-centered experience by, e.g., adjusting one or more visualizations based on an identification of a user. For example, a visualization unit can change a selection of one or more objects being displayed based an identity and associated interests of a detected user. A visualization unit can change a price or identifier of one or more items, e.g., offering sales, based on user identification. A unit can automatically dispense scents associated with one or more goods, or designed to be pleasing to a user, e.g., in response to detecting a user is approaching a unit. In some cases, one or more predictive machine learning models can be used to predict an approaching user, e.g., to reduce energy usage required in dispensing the scent or scent reservoirs used.

In some cases, techniques described can reduce a carbon footprint of a store. For example, this specification describes contextually aware and activated elements that can adjust power consumption, e.g., by changing operating states, in response to detecting one or more actions in an environment, such as one or more actions by people viewing a visualization unit. This specification describes a slim profile of a visualization unit that can reduce a physical footprint of a store which can reduce energy consumption by reducing actions that are square footage dependent, e.g., store lighting, air conditioning, manufactured element, among others. This specification describes visualization of objects, using a visualization unit, which may otherwise be shipped to, and stored, physically within a store. By using the visualization unit, shipments to the store can be reduced or eliminated. Subsequent shipment to users can produce less carbon emissions than shipments to the store—e.g., based on the number of items shipped to a requesting user and subsequently becoming spoiled or not used compared to the number of items that become spoiled or not used that are typically shipped to, or stored at, a retail location. Techniques described can enable stores to exist in areas that may not traditionally provide sufficient demand across one or more products. By removing physical storage of products, stores can be located in these areas without stored product spoilage, e.g., between requests for products, which can be sufficient to either preempt the functioning of a store location or may limit the products to shelf stable options.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example visualization unit.

FIG. 2 is a flowchart of an example process for a visualization unit.

FIG. 3 shows a second example visualization unit.

Like reference numbers and designations in the various drawings indicate like elements.

### DETAILED DESCRIPTION

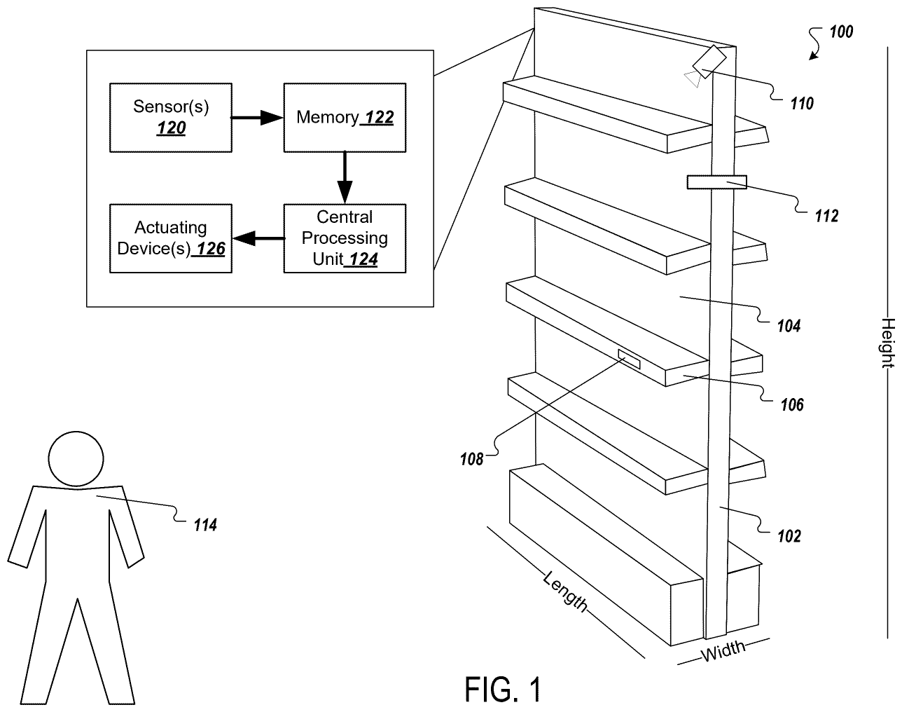

FIG. 1 shows an example visualization unit 100. The visualization unit 100 can be used to help visualize objects. In some cases, visualization units can be used to visualize objects available for purchase in a store without having associated objects in stock. The visualization unit 100 can receive requests for an object and can generate a delivery instruction to deliver the object to an address of a requesting user. By not having to stock items on location, visualization units can reduce spoilage and store square footage.

The visualization unit 100 includes a standing wall 102, a first screen 104, an ornamental shelf 106, and a digital label 108. The visualization unit 100 can include one or more actuating devices. Actuating devices can include sensors, such as a camera 110 or proximity sensor, or a scent dispenser 112. In some cases, actuating devices are embedded within the visualization unit 100, e.g., to reduce clutter or to prevent theft. For example, the camera 110 can be embedded within the standing wall 102, such as a top portion of the standing wall 102. The standing wall 102 can include a window to allow the camera 110 to view outside the standing wall 102 but remained housed within the standing wall 102. The scent dispenser 112 can be embedded in the standing wall, e.g., with a screen or other permeable barrier that allows a scent compound to be dispensed from within the standing wall 102.

In some implementations, the visualization unit 100 has a height between 150 and 250 cm, a length between 50 and 150 cm, and a width between 15 and 50 cm. For example, the visualization unit 100 can have a height of 220 cm, a length of 100 cm, and a width of 25 cm. Although shown with the first screen 104 opposite a second screen (not shown), a visualization unit can be one sided, e.g., having only one screen. In some cases, the ornamental shelf 106 is not used.

In some implementations, the ornamental shelf 106 extends far enough to appear as a shelf while limiting blocking of a screen, such as the first screen 104. For example, the ornamental shelf 106 can be sufficiently narrow to prevent blocking a line of sight between a person, such as the person 114 and a portion of the first screen 104 either above or below the ornamental shelf 106. In general, wider shelves may block a line of sight to a portion of screen below the shelf when a person is close and looking down. Similarly, wider shelves may block a line of sight to a portion of screen above the shelf when a person is close and looking up. To help ensure that a person, such as the person 114, can view various areas of the first screen 104, the ornamental shelf 106 can be narrow—e.g., less than or equal to one half, one quarter, or one third of the height of the portion of screen above the shelf or below the shelf. For example, the ornamental shelf 106 may protrude a distance of fifteen centimeters from the standing wall 102 while the portion of the first screen 104 above or below the ornamental shelf 106 may extend to a height of thirty centimeters. In this case, the ornamental shelf 106 would be one half the height of the portion of the first screen 104. In some cases, other ratios or measurements are used. Traditional product shelving may have a larger ratio of width to height, e.g., to help store all of the physical products. This additional width for storage prevents compact shelving and compact stores offering a variety of products, e.g., by increasing the necessary width of aisles. With the described techniques, aisles can be more compact which can create more compact stores that can be located in more areas and can reduce energy use related to lighting or air conditioning.

In some implementations, the visualization unit 100 can be constructed with an aluminum frame. The ornamental shelf 106 on the visualization unit can include a chip board and light emitting diode (LED) strips. In some implementations, at least a portion of the first screen 104 is printed, e.g., using recyclable ink. Film of the first screen 104 can include polyvinyl chloride (PVC) screen film. The first screen 104 can include a liquid crystal display (LCD), such as ultrathin LCD, light emitting diode display (LED), organic LED, or a combination of these, among others. The first screen 104 can include a user interface, e.g., providing a user with an interface to request a displayed object, ask an object-related question, or adjust aspects of a display. A user interface can include a touch screen, microphone, or camera. A touch screen can be used by a user to purchase displayed objects.

In some implementations, the visualization unit 100 includes a display screen. For example, the first screen 104 can display an item for sale. The visualization unit 100 can replicate a look and feel of a traditional supermarket, or other store, display shelf, e.g., without requiring actual goods or stock rooms located on a premises.

In some implementations, the visualization unit 100 includes digital displays. For example, a unit can use the digital label 108 to display identifiers. The digital label 108 can be positioned on an outer edge of the ornamental shelf 106, as shown, or in another location, such as on the first screen 104. Identifiers can include one or more of: a name of an object displayed by the visualization unit, a price of an object, object specific scanning information (e.g., QR code or barcode), object description, object size, object quantity, price per unit (e.g., for some types of objects, such as fruit, vegetables, fish, chicken, or others), special offers and relevant scanning information (e.g., QR code or barcode), out of stock information, or a combination of these. In some cases, digital identifiers can be communicably connected to processors configured to change their values, e.g., in response to one or more input data. Input data can include requests for changing a name or price, past purchases of an item, expected purchases within a time frame, a current person viewing the visualization unit, an area where the visualization unit is located, among others. In some cases, price changes and out of stock events can be represented by digital identifiers displayed on the visualization unit, e.g., using the digital label 108.

In some implementations, the visualization unit 100 includes one or more actuating devices, e.g., for built-in monitoring, energy capturing, operational technologies, or a combination of these among others. For example, the visualization unit 100 can include the camera 110, the scent dispenser 112, connected energy capturing devices (not shown), proximity sensors (not shown), or a combination of these among others. Energy capturing devices can include cadmium telluride ultra-thin transparent film.

In some implementations, one or more cameras, such as the camera 110 can be connected with one or more processers that perform processing using one or more trained models. For example, cameras can capture images of a person 114 within a sensing threshold of the visualization unit 100. One or more processors can track motion, such as eye motion, of the person 114 based on the captured images. Tracking motion can include tracking the motion of one or more people as they move within a store that includes one or more visualization units.

In some implementations, the visualization unit 100 is placed within a building or outside. The visualization unit 100 can be activated to display one or more objects when a person, such as the person 114, is detected by the visualization unit within a viewing distance. The visualization unit 100 can be placed alongside other visualization units. A single visualization unit can display the same or different objects. Adjacent visualization units can display the same or different objects. A visualization unit can be adjacent to tangible objects displayed for purchase, e.g., a traditional grocery store shelf or other product placement. In some cases, the visualization unit 100 is used only for some products within a store, e.g., products that quickly spoil, are not frequently purchased, are at risk of theft, e.g., because of per unit price, or other reasons.

In some implementations, a user interacts with the visualization unit 100. For example, a user can view objects displayed on a visualization unit, interact with the display to request objects for delivery, ask object-related questions, analyze information related to the object, among other actions. In some cases, a user can use a smartphone or other computer to interact with the visualization unit 100. For example, a user can download an application configured to interact with one or more visualization units. A user can register an application by a variety of means, e.g., using mobile phone number, email address, social media account, or the like. One-time passcodes (OTP) or the like can be used during registration, e.g., to uniquely tie a user with an instance of an application.

In some implementations, the visualization unit 100 displays objects that are available for viewing or purchase on the internet. For example, a user can use an application running on a smartphone or computer to shop online, e.g., for one or more grocery store items. A user can add items to a virtual cart either by navigating on a webpage or application running on a computer or by navigating a physical space equipped with one or more visualization units. After all objects are added to the virtual cart, a user can check-out.

In some implementations, the standing wall 102 houses one or more wires for operating one or more devices of the visualization unit 100. For example, the standing wall 102 can house a power cable connecting the camera 110 to a power source, such as a renewable power energy source. The standing wall 102 can house a cable configured to receive data from the camera 110 or actuate the camera, e.g., pan, zoom, turn off, wake, sleep, or a combination of these among other actions.

In some implementations, the standing wall 102 houses a scent compound for the scent dispenser 112. For example, the scent dispenser 112 can be used to dispense scents associated with one or more objects displayed on the first screen 104. The standing wall 102 can include one or more scents for use by the scent dispenser 112. In some cases, one or more computers can receive data indicating a current object being displayed on the first screen 104 and adjust a scent reservoir to be used by the scent dispenser 112. In response to receiving data indicating a currently displayed object, the one or more computers of the visualization unit 100 can adjust a reservoir used by the scent dispenser 112 to adjust a corresponding scent dispensed by the scent dispenser 112. In some cases, the scent dispenser 112 is configured to dispense scent in response to motion sensor data. Motion sensor data can be generated by one or more motion sensors, e.g., affixed to a visualization unit or communicably connected to a visualization unit. The one or more motion sensors can be placed at an end point of an aisle. The scent dispenser 112 can be configured to dispense scent in response to determining, e.g., by the visualization unit 100, that a person is moving toward the scent dispenser 112.

In some implementations, a user profile is used for in person object viewing with the visualization unit 100. For example, a user profile can include one or more items of information, such as a name, date of birth, address, log-in preference, dietary preferences or requirements, payment methods or preferred payment method, or facial recognition registration.

In some implementations, the visualization unit 100 performs one or more adjustments using received sensor data. For example, the visualization unit 100 can include sensor(s) 120, computer memory 122, a central processing unit (CPU) 124, and actuating device(s) 126. The sensor(s) 120—such as the camera 110, proximity sensor, or the scent dispenser 112—can obtain sensing data and provide the data to the memory 122. The CPU 124 can receive data from the memory 122 can determine whether one or more conditions have been satisfied. In response to one or more conditions being satisfied, the CPU 124 can generate a signal to control or adjust one or more actuating device(s) 126, such as the scent dispenser 112.

In some cases, displays of objects on the visualization unit 100 can be adjusted based on information in a user profile. For example, the sensor(s) 120 can obtain one or more images of the person 114 within a viewing distance, or prior to being within a viewing distance of the visualization unit 100. The images can be stored in the memory 122, such as random access memory (RAM). The CPU 124 can process the images stored in the memory 122 to determine whether or not the person 114 is approaching the visualization unit 100 or is within a viewing threshold distance. In response to determining one or both of these conditions are satisfied, the CPU 124 can generate a signal and transmit the signal to the actuating device(s) 126 to adjust one or more displays based on user profile data for the specific detected user.

In some cases, adjustments can include not displaying age restricted items based on a date of birth, e.g., changing a display element for alcohol to another, non-alcohol object, in response to detected that a user within a threshold distance from the visualization unit 100 is below a legal age for alcohol purchases.

In some implementations, a user interacts with the visualization unit 100 using identity recognition or via mobile device. For example, the visualization unit 100 can personalize interactions by detecting and identify a user, e.g., using facial recognition, iris scanning, gait detection, among other identifying techniques. In some cases, a user may opt out of identity recognition interactions and use a mobile device. The mobile device can use short wave or long wave radio frequencies to communicate with elements of the visualization unit, such as the sensor(s) 120, wherein the sensor(s) 120 include a modem configured to receive signals from one or more registered mobile devices.

In some implementations, identity recognition is used to control access to an area that houses one or more visualization units. For example, a user, such as the person 114, can approach an area that houses one or more visualization units and be detected by one or more cameras. The cameras can be configured to provide image data to one or more object detection and identity recognition machine learning models. A user can be identified by one or more computers operating on received images of the user and operating one or more facial recognition, iris scanning, appearance detection, or other detection processes. One or more computers can use the CPU 124. If the user is a verified existing user, the CPU 124 can generate a signal configured to activate devices for access control, such as gates, to the area and transmit the signal to the devices.

In some cases, instead of or in addition to identity recognition, a user activates a screen on a mobile device to display a scanning element, such as a QR code or bar code. For example, the person 114 can present the scanning element shown on a display of a mobile device to one or more scanners at an entrance to an area that houses one or more visualization units. The scanned data can be sent to one or more computers, such as a control unit, configured to process the scanned data. Similar to the facial recognition entrance process, in response to the one or more computers determining that the scanning element satisfies one or more validity checks, the one or more computers can generate a signal configured to activate devices for access control, such as gates, to the area and transmit the signal to the devices to cause the devices to open.

In some implementations, kinetic flooring is used in an area adjacent to one or more visualization units. Kinetic flooring can use the energy of people walking to generate electricity. Electricity generated by kinetic flooring can be used, at least in part, to power one or more visualization units. Energy can be stored in energy storage and used as need to power one or more visualization units or related processes.

In some implementations, footsteps of users are tracked. For example, the sensor(s) 120 can include one or more pressure sensors or cameras tracking movement of people. The CPU 124 can receive data indicating footsteps of one or more users. The CPU 124 can generate data representing routes taken by users through an area housing one or more visualization units, heat maps of user activity, waiting times at specific regions within an area, or the like. The CPU 124 can generate data representing: energy created by one or more people within an area, e.g., of a store; a habit, pattern, impulse purchases, or other features of one or more people within the area; or a combination of these among others.

In some implementations, scent dispensers, such as the scent dispenser 112, are used to provide user activated scents within regions of one or more visualization units, such as the visualization unit 100. For example, as a user walks through an area that houses one or more visualization units, one or more sensors of the visualization unit 100 can provide data to the CPU 124 of the visualization unit 100. The CPU 124 can detect a user based on the sensor data and generate a signal configured to cause one or more scent dispensers to release a particular scent. The CPU 124 can transmit the generated signal to the actuating device(s) 126, which can include the scent dispenser 112, to cause the dispersion of scent material, e.g., compound configured to smell similar to an object being displayed by the visualization unit. Scents can include fruits, tea, coffee, bread, condiments, personal care, detergent, or the like.

In some implementations, scent dispensers only release scents in the presence of one or more users. For example, the visualization unit 100 can include one or more sensors, such as the sensor(s) 120, configured to detect a presence of one or more users. The visualization unit 100 can include one or more computers using the CPU 124 configured to receive data from the one or more sensors and perform operations in response to processing of the received data.

In some implementations, scent dispensers do not release scents for one or more users, e.g., based on an identification of the one or more users. For example, one or more users can indicate in a user profile that they prefer not to have artificial scents dispensed while near a visualization unit, such as the visualization unit 100. One or more computers operating on data provided by sensors of a visualization unit can detect an identity of a user within a sensing distance, e.g., a sensing distance of a given sensor. In some cases, one or more trained models of the one or more computers are configured to predict, based on movement of the individual and training data indicating users approaching one or more visualization units, whether or not a user is going to approach one or more visualization units. In response to sensing a user, or predicting a user is likely to approach, the one or more computers can identify the user, e.g., using biometric information like facial recognition, or by sending or receiving signals from a mobile device of the user. The one or more computers can use an identity of the user to access one or more profile information data items of the user, e.g., stored in the memory 122. Based on one or more profile information data items of the user indicating that the user prefers no scent activation, the visualization unit 100 can send a signal to the scent dispenser 112 to not activate for a given time period or until the one or more computers send a second signal configured to cause a scent dispenser to resume normal operation and disperse scents.

Disabling scent dispenser can be referred to as a user identity-based adjustment. Other adjustments that can be performed by the visualization unit 100 include changing a displayed price or description of an object based on a presence or identity of an approaching person. Adjustments can include increasing or decreasing a level of scent dispensed. Adjustments can include changing a displayed object, e.g., by actuating printed panels of the first screen 104 or adjusting a digital display of the first screen 104.

The person 114 can approach the visualization unit 100 and look at objects shown on the visualization unit 100. The camera 110 can capture images representing the person 114, e.g., body movement, eye movement, or a combination of these among others. The visualization unit 100 can store data related to the person 114, e.g., profile data or sensor data, in the memory 122. The visualization unit 100 can identify a profile that matches the data captured for the person 114—e.g., by matching facial recognition data, matching a signal identifier included in a signal from a device of the person 114, or scanning a scanning element provided by the person, such as a QR code on a display of a smartphone.

The person 114 can select a quantity for an item displayed on the visualization unit 100. For example, the visualization unit 100 can display an object A. The person 114 can interact either with an interface of the visualization unit 100 or a mobile device communicably connected with the CPU 124, e.g., by one or more servers. In some cases, the person 114 can use an application or webpage to select an object that is displayed on the visualization unit 100. One or more objects can be added to a virtual basket.

In some implementations, dietary preferences or restrictions can be checked after or before user selection of objects. For example, at a time that a product is added to a basket, the CPU 124, or other computer communicably connected to the CPU, such as one or more cloud compute servers, can check for any dietary preferences or restrictions provided by the person 114, e.g., by using an identifier of the person 114 to query a database of dietary preferences or restrictions keyed by user identifier. The one or more computers can determine if any object added to the basket includes items on, or not on, the dietary preferences or restrictions. In some cases, the one or more computers can generate preemptive notifications to the person 114 if the person 114 is looking at an object or if the person 114 queries the one or more computers using an object-based question, such as “can I eat this?” while standing in front of a display of a particular object to be queried by the one or more computers against dietary preferences or restrictions of the person 114. The person 114 can override a notification for violations of dietary preferences or restrictions.

In some implementations, the person 114 adds objects from multiple different visualization units, including the visualization unit 100, to the same virtual basket. For example, the person 114 can approach the visualization unit 100 and select an object displayed on the first screen 104 and identified with the digital label 108. The CPU 124 can obtain data representing data of an interaction of the person 114 and save the data within the memory 122. The CPU 124 can save the interaction data indicating a selection of one or more objects at the visualization unit 100 within a memory allocated for the storage of a virtual basket of the person 114. If the person 114 moves to another visualization unit, such as an adjacent visualization unit, the CPU 124 can receive additional interaction data and save the additional interaction data to the memory 122 to store additional adjustments to the virtual basket.

In some implementations, the CPU 124 controls one or more visualization units. In some implementations, the CPU 124 controls only the visualization unit 100 but is communicably connected with one or more computers that control one or more other visualization units. For example, the CPU 124 can receive sensor data representing approaching users or interactions of users and provide the data to one or more computers, or generate signals configured to cause the one or more computers to adjust one or more stored memories or cause one or more actuating devices to operate, such as the actuating device(s) 126.

In some implementations, the person 114 checks out with a mobile device. For example, after adding one or more objects to a cart using a mobile device of one or more visualization units, the person 114 can use their mobile device to check-out. The mobile device can be equipped with a webpage or application configured with interfaces to pay or schedule deliveries. The person 114 can indicate whether they prefer instant delivery or scheduled delivery.

In some implementations, one or more computers predicts a delivery option for the person 114. Predicted delivery options can be transmitted to a mobile device of the person 114. The delivery options signal transmitted to the mobile device can be configured to cause the mobile device to display an interface for the person 114 to confirm a prediction or deny a prediction. In some cases, one or more machine learning models are used to predict a delivery option for the person 114. For example, a machine learning model can be trained on user data related to prior delivery instructions received from the person 114 or received from users with characteristics similar to person 114. Training data can include data indicating a particular user and a delivery option selected by that user. One or more machine learning models can predict that delivery option selected by the user. One or more computers can be configured to train the one or more machine learning models by comparing the predicted delivery option with the known delivery option selected by the user. Based on the comparison, the one or more computers can adjust one or more values or parameters of the one or more machine learning models.

In some implementations, one or more machine learning models are trained to create or optimize: product profiles, such as description, size, or other features of a product; product image gallery; product ingredients; nutritional facts; dietary information; or a combination of these among others. One or more machine learning models can learn about shopping habits, product preferences, or other features of a person within a sensing range. The models can provide users with output indicating, e.g., personalized product promotions or recommendations. The output can be specific to requirements provided by a user, e.g., dietary restrictions, preferences, among others. Output can be provided to a user by the CPU 124 or another element of, or communicably connected to, the visualization unit 100. Output can be provided to the user at a specific time, e.g., when the visualization unit 100 or connected component detects the person walking near an area that displays a given product. Output can be provided to the user using a notification on a mobile device of the user, via a display of the visualization unit 100, speakers, holographic display, or a combination of these among others. In some cases, if an output is not detected to have been noticed by a user, e.g., by monitoring actions of the user or monitoring expected response from the notification, alternative notifications can be sent, e.g., using different means. In some cases, a hierarchy of notification techniques are used to sequentially attempt different notification methods until a user is determined to have noticed the notification.

In some implementations, the person 114 approaches the visualization unit 100 and selects one or more objects using an interface displayed on the visualization unit 100. For example, the person 114 can select an object by touching a touch screen included on the first screen 104. In some cases, responsive to a detected touch by the person 114, the visualization unit 100 can display one or more additional interface options, such as plus or minus buttons used to adjust the quantity of the object or an “add to basket” button.

In some implementations, the visualization unit 100 is configured to perform facial recognition to uniquely identify a user. For example, the visualization unit 100 can be configured to use the camera 110 to capture images of the person 114. The captured images of the person 114 can be processed by the CPU 124. The CPU 124 can be configured to perform one or more object detection or identification processes. The CPU 124 can perform one or more processes to detect and identify the person 114. After identifying the person 114, selections made by the person 114 using the visualization unit 100 can be added, by the CPU 124, to a virtual basket uniquely associated with the person 114.

In some implementations, the person 114 presents a scanning element which is used by the visualization unit 100 to uniquely identify the person 114. For example, the person 114 can you use a smartphone or other mobile device to display a scanning element, such as a QR code. The visualization unit 100 can include a scanner configured to scan a scanning element displayed by the person 114. A scanning element provided by the person 114 can uniquely identify the person 114. The CPU 124 can receive input data from a sensor, such as a scanner, and use the input data to uniquely identify the person 114. In response to uniquely identifying the person 114, the CPU 124 can add data representing one or more objects selected by the person 114 using an interface of the visualization unit 100 to a virtual basket associated with the person 114.

In some implementations, a person who has not yet been uniquely identified, either by identity recognition or scanning element, can make selections using an interface of the visualization unit 100. If a not yet identified person makes a selection, the CPU 124 can add associated data to a memory location, e.g., included in the memory 122, allocated for an unknown person. After the visualization unit 100 identifies an unknown person with associated data allocated in memory, the CPU 124 can adjust the memory to add a unique identifier specific to the identified previously unknown person.

In some implementations, the visualization unit 100 displays a scanning element uniquely associated with one or more objects displayed on the visualization unit 100. For example, the visualization unit 100 can display a QR code corresponding to a first object. The person 114 can use a mobile device, such as a smartphone, to scan the displayed QR code. In response to scanning the displayed QR code, the scanning device used by the person 114 can display additional interfaces. Additional interfaces can include a plus or minus button to adjust quantity or an “add to basket” button. In some cases, a mobile device used by the person 114 can check added items against dietary restrictions or preferences specified by the person 114. Dietary restrictions or preferences can be specified in a user profile or by other means. If the mobile device determines that one or more items added to a basket are items that are restricted or are not on a list of dietary preferences, the mobile device can generate a notification and provide the notification to a display element of the mobile device. The notification can include information of the object or of the dietary restrictions or preferences. The notification can allow the person 114 to override and add an object anyway, even if the object is on a dietary restriction list.

In some implementations, the person 114 can check out by going to a virtual check out gate. A virtual checkout gate can include a camera that can detect the person 114. The virtual checkout gate can include a display element configured to display the virtual basket associated with the person 114. In some cases, the virtual checkout gate performs identity recognition, such as facial recognition, on the person 114. In some cases, the CPU 124 generates a signal and provides it to a communication element, such as a modem, which can be included in the actuating device(s) 126. The communication element can transmit a wireless or wired communication signal, representing the generated signal by the CPU 124, to the virtual checkout gate. The transmitted signal can indicate a current location of the person 114, an identity of the person 114, a virtual basket of the person 114, or a combination of these among other data related to the person 114. By transmitting the signal, the CPU 124 can reduce processing bandwidth at the virtual checkout gate, e.g., by preventing additional identity recognition processes being performed at the virtual checkout gate.

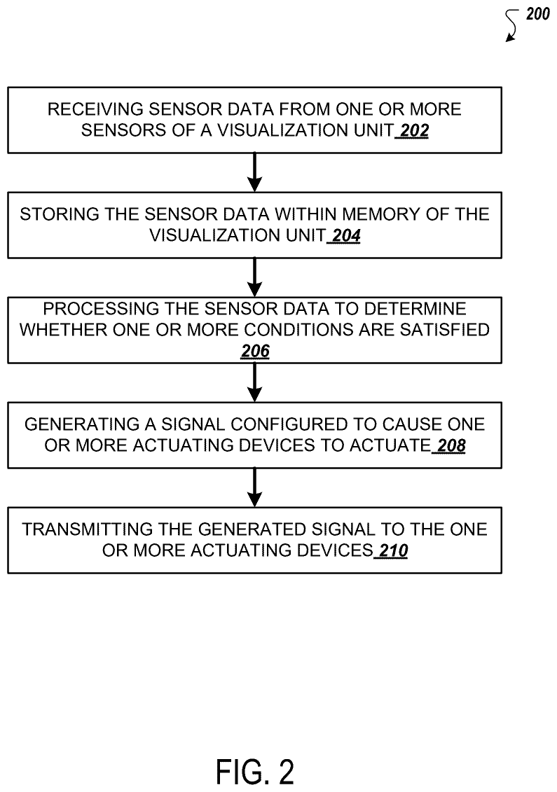

FIG. 2 is a flowchart of an example process 200 for a visualization unit. For convenience, the process 200 will be described as being performed by a system of one or more computers, located in one or more locations, and programmed appropriately in accordance with this specification. For example, a visualization unit, e.g., the visualization unit 100 of FIG. 1, or one or more computers communicably connected to the visualization unit, appropriately programmed, can perform the process 200.

The process 200 includes receiving sensor data from one or more sensors of a visualization unit (202). For example, the visualization unit 100 can be equipped with one or more sensors, such as the camera 110 or sensors of the sensor(s) 120. The sensors of the visualization unit 100 can receive sensor data, such as the sensor data representing the person 114.

The process 200 includes storing the sensor data within memory of the visualization unit (204). For example, the visualization unit 100 can include one or more memory units within its body, e.g., such as the memory 122. In some cases, the memory 122 is housed within the standing wall 102 or another part of the visualization unit 100. In some cases, memory units can be included as part of one or more sensors, such as the camera 110. In some cases, memory units can be communicably connected to the visualization unit 100, such as cloud storage or other storage systems.

The process 200 includes processing the sensor data to determine whether one or more conditions are satisfied (206). For example, the visualization unit 100 can process sensor data. In some cases, the CPU 124 is configured to process sensor data received from the sensor(s) 120. The sensor data can be stored in the memory 122. Conditions can include conditions related to one or more detected persons or other events detected within a sensing proximity of the visualization unit 100.

The process 200 includes in response to determining that one or more conditions are satisfied, generating a signal configured to cause one or more actuating devices to actuate (208). For example, the CPU 124 can generate one or more commands configured to cause the actuating device(s) 126 to actuate. The actuating device(s) 126 can include a combination of one or more devices, such as the scent dispenser 112, a self-cleaning device, the digital label 108, the camera 110, the first screen 104, one or more elements of another visualization unit, among others.

The process 200 includes transmitting the generated signal to the one or more actuating devices (210). For example, the CPU 124 can send one or more generated signals to the actuating device(s) 126. The signals can be configured to cause performance of actions by the actuating device(s) 126 immediately or after a delay specified by the one or more generated signals. In some cases, the CPU 124 transmits a signal to an intermediary device that approves or denies one or more actions, e.g., based on one or more sets of criteria or processes.

In some cases, the CPU 124 can send a signal to reduce energy consumption of one or more devices in response to detecting one or more conditions via sensor data. For example, the CPU 124 can send a signal to dim or turn off one or more lights or displays, such as the digital label 108 or the first screen 104. By a sending a signal to dim or turn off one or more lights or displays, the CPU 124 can reduce energy consumption, e.g., of a store that includes one or more visualization units. In some cases, the CPU 124 sends a signal to reduce energy consumption of one or more devices in response to detecting that there are no persons detected in a given area, a store is empty, or a time condition is satisfied.

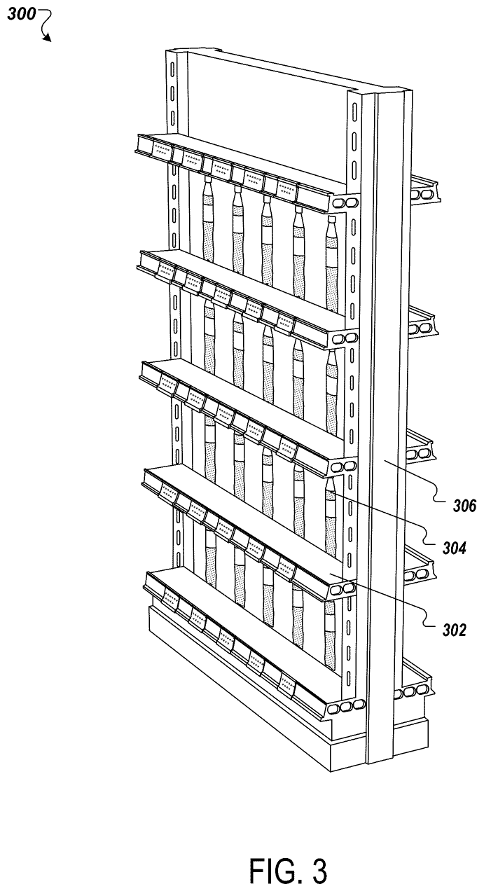

FIG. 3 shows a second example visualization unit 300. The visualization unit 300 includes ornamental shelving 302 and a first screen 304 coupled to a standing wall 306. Aspects of the visualization unit 100 can be included on the visualization unit 300 and vice versa. In some cases, actions performed by elements of the visualization unit 100 can be performed by similar elements of the visualization unit 300 (not shown). For exemplary size indication, the visualization unit 300 includes rows of displayed soda bottles, which can be a type of object displayed by a visualization unit, such as the visualization unit 300.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The subject matter and the actions and operations described in this specification can be implemented in digital electronic circuitry, in tangibly-embodied computer software or firmware, in computer hardware, including the structures disclosed in this specification and their structural equivalents, or in combinations of one or more of them. The subject matter and the actions and operations described in this specification can be implemented as or in one or more computer programs, e.g., one or more modules of computer program instructions, encoded on a computer program carrier, for execution by, or to control the operation of, data processing apparatus. The carrier can be a tangible non-transitory computer storage medium. Alternatively or in addition, the carrier can be an artificially-generated propagated signal, e.g., a machine-generated electrical, optical, or electromagnetic signal, that is generated to encode information for transmission to suitable receiver apparatus for execution by a data processing apparatus. The computer storage medium can be or be part of a machine-readable storage device, a machine-readable storage substrate, a random or serial access memory device, or a combination of one or more of them. A computer storage medium is not a propagated signal.

The term “data processing apparatus” encompasses all kinds of apparatus, devices, and machines for processing data, including by way of example a programmable processor, a computer, or multiple processors or computers. Data processing apparatus can include special-purpose logic circuitry, e.g., an FPGA (field programmable gate array), an ASIC (application-specific integrated circuit), or a GPU (graphics processing unit). The apparatus can also include, in addition to hardware, code that creates an execution environment for computer programs, e.g., code that constitutes processor firmware, a protocol stack, a database management system, an operating system, or a combination of one or more of them.

A computer program can be written in any form of programming language, including compiled or interpreted languages, or declarative or procedural languages; and it can be deployed in any form, including as a stand-alone program, e.g., as an app, or as a module, component, engine, subroutine, or other unit suitable for executing in a computing environment, which environment may include one or more computers interconnected by a data communication network in one or more locations.

A computer program may, but need not, correspond to a file in a file system. A computer program can be stored in a portion of a file that holds other programs or data, e.g., one or more scripts stored in a markup language document, in a single file dedicated to the program in question, or in multiple coordinated files, e.g., files that store one or more modules, sub-programs, or portions of code.

The processes and logic flows described in this specification can be performed by one or more computers executing one or more computer programs to perform operations by operating on input data and generating output. The processes and logic flows can also be performed by special-purpose logic circuitry, e.g., an FPGA, an ASIC, or a GPU, or by a combination of special-purpose logic circuitry and one or more programmed computers.

Computers suitable for the execution of a computer program can be based on general or special-purpose microprocessors or both, or any other kind of central processing unit. Generally, a central processing unit will receive instructions and data from a read-only memory or a random access memory or both. The essential elements of a computer are a central processing unit for executing instructions and one or more memory devices for storing instructions and data. The central processing unit and the memory can be supplemented by, or incorporated in, special-purpose logic circuitry.

Generally, a computer will also include, or be operatively coupled to, one or more mass storage devices, and be configured to receive data from or transfer data to the mass storage devices. The mass storage devices can be, for example, magnetic, magneto-optical, or optical disks, or solid state drives. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

To provide for interaction with a user, the subject matter described in this specification can be implemented on one or more computers having, or configured to communicate with, a display device, e.g., a LCD (liquid crystal display) monitor, or a virtual-reality (VR) or augmented-reality (AR) display, for displaying information to the user, and an input device by which the user can provide input to the computer, e.g., a keyboard and a pointing device, e.g., a mouse, a trackball or touchpad. Other kinds of devices can be used to provide for interaction with a user as well; for example, feedback and responses provided to the user can be any form of sensory feedback, e.g., visual, auditory, speech, or tactile feedback or responses; and input from the user can be received in any form, including acoustic, speech, tactile, or eye tracking input, including touch motion or gestures, or kinetic motion or gestures or orientation motion or gestures. In addition, a computer can interact with a user by sending documents to and receiving documents from a device that is used by the user; for example, by sending web pages to a web browser on a user's device in response to requests received from the web browser, or by interacting with an app running on a user device, e.g., a smartphone or electronic tablet. Also, a computer can interact with a user by sending text messages or other forms of message to a personal device, e.g., a smartphone that is running a messaging application, and receiving responsive messages from the user in return.

This specification uses the term “configured to” in connection with systems, apparatus, and computer program components. That a system of one or more computers is configured to perform particular operations or actions means that the system has installed on it software, firmware, hardware, or a combination of them that in operation cause the system to perform the operations or actions. That one or more computer programs is configured to perform particular operations or actions means that the one or more programs include instructions that, when executed by data processing apparatus, cause the apparatus to perform the operations or actions. That special-purpose logic circuitry is configured to perform particular operations or actions means that the circuitry has electronic logic that performs the operations or actions.

The subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some implementations, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of what is being claimed, which is defined by the claims themselves, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claim may be directed to a subcombination or variation of a subcombination.

Similarly, while operations are depicted in the drawings and recited in the claims in a particular order, this by itself should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A visualization unit comprising:
a standing wall disposed between a first screen and a second screen, wherein the first screen covers a first face of the standing wall and the second screen covers a second face of the standing wall, wherein the first face is opposite the second face;
the standing wall having ornamental shelving partially obscuring the first screen and the second screen and defining rows along the first screen and the second screen;
an actuating device mechanically coupled to the standing wall; and
a digital label along an outer edge of the ornamental shelving configured to display an identifier of an object represented on an unobscured portion of either the first screen or the second screen.

2. The visualization unit of claim 1, wherein the actuating device is mechanically coupled to the standing wall along an outer frame of at least one of the first face of the second face of the standing wall.

3. The visualization unit of claim 1, wherein the actuating device includes at least one of a scent dispenser, a motion detector, a communication element, or a camera.

4. The visualization unit of claim 1, wherein the ornamental shelving does not hold objects displayed on the first screen.

5. The visualization unit of claim 1, wherein the standing wall houses one or more elements providing electricity or resources to the actuating device.

6. The visualization unit of claim 5, wherein the standing wall houses one or more scent reservoirs used by a scent dispenser.

7. The visualization unit of claim 1, wherein the actuating device is controlled by one or more computers configured to receive image data, process the image data to determine whether a person is approaching the visualization unit, and, in response to determining that a person is approaching the visualization unit, transmit a signal to the actuating device to cause the actuating device to disperse a scent.

8. The visualization unit of claim 1, wherein the actuating device is embedded within the standing wall.

9. The visualization unit of claim 8, wherein the standing wall includes a permeable cover to house the actuating device within the standing wall.

10. A method comprising:
receiving sensor data from one or more sensors of a visualization unit;
storing the sensor data within memory of the visualization unit;
processing the sensor data to determine whether one or more conditions are satisfied;
in response to determining that one or more conditions are satisfied, generating a signal configured to cause one or more actuating devices to actuate; and
transmitting the generated signal to the one or more actuating devices.

11. The method of claim 10, wherein receiving sensor data from the one or more sensors of the visualization unit comprises:
receiving image data from a camera mechanically coupled to the visualization unit.

12. The method of claim 10, wherein receiving sensor data from the one or more sensors of the visualization unit comprises:
receiving proximity data from a proximity sensor mechanically coupled to the visualization unit.

13. The method of claim 10, wherein receiving sensor data from the one or more sensors of the visualization unit comprises:
receiving a signal transmitted by a mobile device.

14. The method of claim 10, wherein receiving sensor data from the one or more sensors of the visualization unit comprises:
scanning a scanning element provided by a mobile device.

15. The method of claim 10, wherein receiving sensor data from the one or more sensors of the visualization unit comprises:
receiving data indicating an interaction of a user selecting an object displayed on the visualization unit.

16. The method of claim 15, wherein storing the sensor data within memory of the visualization unit comprises:
storing the received data within data representing a virtual basket associated with the user.

17. The method of claim 10, wherein generating the signal configured to cause the one or more actuating devices to actuate comprises:
generating a signal configured to cause a scent dispenser to dispense a scent compound.

18. The method of claim 17, wherein generating the signal configured to cause the scent dispenser to dispense the scent compound comprises:
generating a signal configured to instruct the scent dispenser to obtain a scent compound from a particular scent reservoir from a set of one or more scent reservoirs housed in the visualization unit.

19. The method of claim 10, wherein generating the signal configured to cause the one or more actuating devices to actuate comprises:
generating a signal configured to cause a cleaning element to clean an area of the visualization unit.

20. The method of claim 19, wherein generating the signal configured to cause the cleaning element to clean the area of the visualization unit occurs in response to detecting a person is not within a distance of the visualization unit.

21. The method of claim 10, wherein generating the signal configured to cause the one or more actuating devices to actuate comprises:
generating a signal configured to change an operating mode of the visualization unit.

22. The method of claim 21, wherein generating the signal configured to change the operating mode of the visualization unit comprises generating a turn off signal or sleep signal.

23. The method of claim 21, wherein generating the signal configured to change the operating mode of the visualization unit occurs in response to determining a current time of day satisfies a threshold time of day for changing the operating mode.
